(19) United States
(12) Patent Application Publication
(10) Pub. No.: US 2007/0086975 A1
(43) Pub. Date: Apr. 19, 2007

(54) Polymeric micellar complexes and drug delivery vehicles thereof

(76) Inventors: Francis Ignatious, King of Prussia, PA (US); Yu Li, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/596,566

(22) PCT Filed: Dec. 17, 2004
(86) PCT No.: PCT/US04/42768

§ 371 Date: Jun. 16, 2006

Related U.S. Application Data
(60) Provisional application No. 60/530,142, filed on Dec. 17, 2003.
(60) Provisional application No. 60/532,045, filed on Dec. 22, 2003.

(51) Int. Cl. A61K 31/77; A61K 31/704; A61K 31/7024; A61K 31/4745

(52) U.S. Cl. 424/78.27; 525/54.2; 424/649; 514/34; 514/37; 514/283

(57) ABSTRACT

Disclosed are complexes of an amphiphilic copolymer, wherein the amphiphilic copolymer has benzoyl sulfonic acid groups on the hydrophobic segment of the copolymer.

### FIELD OF THE INVENTION

The present invention relates to water soluble amphiphilic block copolymers capable of forming polymeric micelles or nanoparticles. These polymeric micelles and nanoparticles are designed to contain benzoyl sulfonic group in the hydrophobic domains of the micelle forming amphiphilic copolymer, such that they can encapsulate water soluble drug molecules, and hence act as delivery vehicles for the same.

### BACKGROUND OF INVENTION

It is generally desirable to provide pharmaceutical actives in formulations targeted to the disease site in order to permit lower dosing, reduce side effects, and/or to improve patient compliance. This may be particularly true in the case of drugs that tend to have unpleasant side effects, especially when used at high doses, such as certain anti-cancer agents.

It is also desirable to provide pharmaceutical actives in formulations which enhance their availability (e.g., to permit minimal dosing, to improve patient compliance). Polymer-therapeutics are gaining wide acceptance as drug delivery systems. Polymer-therapeutics involve the use of polymeric systems to enhance the drug's circulation half-life and to reduce its toxicity. These characteristics are demonstrated by polyethylene glycol(PEG)conjugated proteins, commonly known as pegylated proteins. An important characteristic of a polymeric bound therapeutic is its passive accumulation at a tumor site, known as the epr effect (enhanced permeability and retention effect), due to the leaky tumor vasculature. This passive targeting is the mechanism of action of an anti-tumor agent, SMANCS, approved in Japan for liver cirrhosis. SMANCS consists of low molecular weight styrene maleic anhydride copolymer conjugated to neocarzinostatin through the anhydride groups present in the polymer. Although the molecular weight of SMANCS is about 16-17 kDa, it forms larger aggregates with serum albumin. The aggregated size of the conjugate, 80 kDa, is said to responsible for the spontaneous but passive accumulation of SMANCS at the tumor site.

Passive targeting mechanism is also exhibited by liposomes, polymeric micelles and nanoparticles having diameters of less than 200 nm. Polymer based nanoparticles and polymeric micelles are formed by spontaneous self assembly of amphiphilic copolymers. These amphiphilic copolymers are composed of hydrophobic and hydrophilic segments, arranged in either block or graft architecture.

Amphipilic block copolymers in aqueous medium undergo micellization by aggregation of the hydrophobic domains. In the case of block copolymers containing ionic and hydrophilic segments, micelle formation is induced by the condensation of the ionic block by oppositely charged molecule or macromolecule.

In vivo, these polymeric micelles can evade the uptake by macrophages and hence exhibit ‘stealth’ characteristics due to the presence of the outer hydrophilic domains. Although many hydrophilic polymers such as polyvinylpyrrolidone, HPMA, chitosan, polyethyleneglycol, can be used as the hydrophilic polymer, PEG is the most frequently used.

Drug molecules may be incorporated into the inner hydrophobic core of the polymeric micelle through hydrophobic association, electrostatic interaction, or chemical conjugation through a labile bond. Electrostatic interaction is the driving force for self-organization into polymeric micelles during the condensation of DNA with block copolymers having hydrophilic cationic segments. In this case, a neutralized polyelectrolyte complex forms the inner core of the micelle, and the outer shell is made up of the hydrophilic segments. Hydrophobic interaction is often used in the solubilization of water insoluble drugs in the hydrophobic domains of polymeric micelles.

Since a majority of the polymeric micellar systems contain PEG as the hydrophilic polymer, the classification of polymeric micelles may be done based on the type of hydrophobic segment in them.

Many known polymeric micellar systems are designed to accumulate at the tumor site passively, due to the size of the delivery vehicle, through the leaky vasculature at the tumor site. It is widely recognized that polymeric micellar systems are capable of encapsulating hydrophobic water insoluble bioactive agents in the inner hydrophobic core by hydrophobic interactions. However, classical polymeric micelles exhibit poor encapsulation efficiency for water soluble bioactive agents. Therefore, there exists a great deal of interest enhancing the encapsulation efficiency of water soluble bioactive agents in polymeric micellar systems.

### SUMMARY OF INVENTION

The present invention relates to complexes of (a) an amphiphilic block or graft copolymer and (b) a water soluble drug containing cationic groups. The amphiphilic block or graft copolymer is functionalized with a benzoyl sulfonic acid group in the hydrophobic segments, such that it can form either ionic or hydrogen bonding interaction with the water soluble cationic drug. The amphiphilic block copolymer can spontaneously self assemble in aqueous medium to form polymeric micelles.

The present invention also relates to a method of forming benzoyl sulfonic acid groups on the amphiphilic polymer, by a reaction in the melt, subsequent to the synthesis of the amphiphilic block copolymer in the melt.

The present invention also relates to such drug delivery vehicles comprising a therapeutic, diagnostic, or prognostic agent (in addition to activity of the antagonist).

### DETAILED DESCRIPTION OF THE INVENTION

In the present invention, incorporation of a benzoyl sulfonic acid moiety into the hydrophobic domain of the amphiphilic block copolymer greatly enhancea the encapsulation efficiency of water soluble cationic drugs in the polymeric micelles. These benzoyl sulfonic acid functionalized polymeric micelles can bind water soluble drugs, endowed with cationic groups, by ionic and/or hydrogen bonding. Moreover, these polymeric micellar complexes can regulate the release of the drug in the biological environment.

The present invention relates to complexes of (a) an amphiphilic block or graft copolymer and (b) a water soluble drug containing cationic groups. The amphiphilic block or graft copolymer is functionalized with a benzoyl sulfonic acid groups in the hydrophobic block, such that it can form either ionic or hydrogen bonding interaction with the water soluble cationic drugs. The amphiphilic block copolymer can spontaneously self assemble in aqueous medium to form polymeric micelles.

In a preferred embodiment, the cationic bioactive agent is complexed to amphiphilic block or a graft copolymer. Suitable amphiphilic block or graft copolymers possess hydrophilic and hydrophobic segments such that th co-polymers can self-assemble to form polymeric micelles in aqueous solution. The size of the polymeric micelles can be suitably engineered by proper selection of the size and nature of the building blocks of the amphiphilic copolymer. The desired sizes of the polymeric micelles are within 200 nm. During the self assembly of the amphiphilic copolymer in water to form polymeric micelles, the outer shell is comprised of the hydrophilic polymer, such that polymeric micelles can evade uptake by the macrophages. Therefore, the polymeric micelles have long circulation half life in the plasma and, due to the small size (<200 nm), can accumulate at the tumor site by epr effect.

According to the present invention, a water soluble bioactive agent complexed to block or graft polymer may be used as such as a delivery system or it may be incorporated into a different polymeric micellar system. Examples of such polymeric micellar systems include block copolymers of polyoxyethylene with polyoxyalkylene, copolymers of polyoxyethylene with poly(alpha-aminoacids) and its derivatives, biodegradable amphipathic copolymers, comprising a hydrophobic biodegradable polymer such as poly(lactic acid)(PLA), poly(glycolic acid)(PGA), polycaprolactone(PC), polyhydroxybutyric acid or polycarbonate coupled to a hydrophilic pharmaceutically acceptable polymers like PEG, polyvinylpyrrolidone, polyvinylalcohol, dextran etc.

In yet another embodiment, it is contemplated that the cationic bioactive agent complexed to amphiphilic graft or block copolymer, may self organize in aqueous medium to form polymeric micelles. The amphiphilic graft and/or block copolymers are made up of hydrophilic and hydrophobic segments. The design and synthesis of these block copolymers are carried out in such way that the hydrophobic polymer segment possess benzoyl sulfonic acid groups which can be used for complexing a water soluble bioactive agent. The complexation of the water soluble cationic bioactive agent may involve either hydrogen bonding or ionic interaction or both. In the absence such a specific interaction between the water soluble bioactive agent and benzoyl sulfonic acid functionalized amphiphilic copolymer, the water soluble bioactive agent could not efficiently encapsulated within the polymeric micelle, and the latter could not be used a delivery system for the former. The hydrophilic polymer segment may be chosen from polyethylene glycol (PEG), polyvinylpyrrolidone (PVP), polyacrylamide (PA), poly(hydroxypropyl acrylamide), polyvinylalcohol (PVA), polysaccharides, polyaminoacids, polyoxazoline, and copolymers and derivatives thereof. Hydrophobic polymer segments may include poly(alpha-hydroxy acids) such as polylactic acid, polycaprolactone, polydioxanone, polycarbonates, polyanhydrides, polyorthoesters, hydrophobic derivatives of poly(alpha-amino acids), such as polylysine, polyaspartic acid, and polyglutamic acid, and polyoxyalkylenes, such as polypropylene oxide, polyoxybutylene etc.

The present invention also provides a novel method of preparing amphiphilic biodegradable polymers having benzoyl sulfonic groups at the hydrophobic terminus, using a single step process, as shown below:

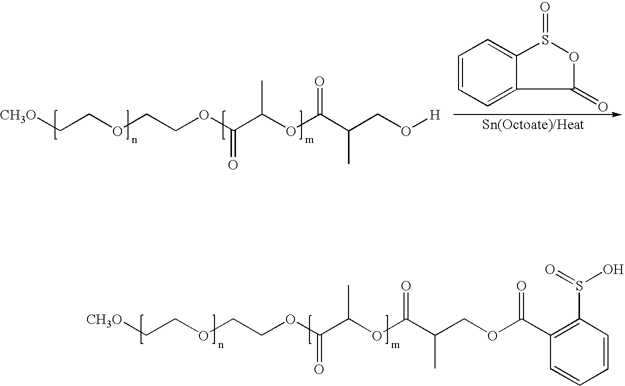

Ring opening polymerization techniques are known in the art and may be employed to prepare the functionalized polymer. The ring opening polymerization; may be carried out either in solution or melt, preferably in a melt. Suitable catalysts are known in the art and are preferably employed. Transition metal catalysts, e.g., stannous octoate, stannous chloride, zinc acetate, zinc, SnO, SnO₂, Sb₂O₃, PbO, and FeCI₃, are preferred, with stannous octoate more preferred. Other examples of suitable catalysts include GeO₂ and NaH. The polymerization reaction temperature will typically be from about 100 to about 200° C. As will be understood by those skilled in the art, the resulting polymer molecular weight will be determined by the molar ratio of the hydrophobic monomer to the hydroxy group present on the alpha methoxy omega hydroxy polyalkylene glycol. The polymer molecular weight will typically be about 40,000 or less, although higher molecular weights may be used. This method of introducing the benzoyl sulfonic acid functional groups onto the biodegradable polymer can be carried out in a melt, subsequent to the ring opening polymerization of the cyclic monomers which provides the biodegradable polyester. This method enables functionalization of the polymer in the melt, without having to isolate the polymer.

The above polymer having benzoyl sulfonic acid groups is used to encapsulate pharmaceutically active agents, by complexation between the anionic sulfonic acid groups on the polymer and the cationic groups on the bioactive agent. Pharmaceutical actives include therapeutic agents and diagnostic agents.

Therapeutic pharmaceutical actives may be selected, for example, from natural or synthetic compounds having the following activities: anti-angiogenic, anti-arthritic, anti-arrhythmic, anti-bacterial, anti-cholinergic, anti-coagulant, anti-diuretic, anti epilectic, anti-fungal, anti-inflammatory, anti-metabolic, anti-migraine, anti neoplastic, anti-parasitic, anti-pyretic, anti-seizure, anti-see, anti-spasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic and vasodilating. The present invention may be especially useful for delivering cytotoxic therapeutic agents.

Examples of therapeutic agents that can be delivered include topoisomerase I inhibitors, topoisomerase VII inhibitors, anthracyclines, vinca alkaloids, platinum compounds, antimicrobial agents, quinazoline antifolates thymidylate synthase inhibitors, growth factor receptor inhibitors, methionine aminopeptidase-2 inhibitors, angiogenesis inhibitors, coagulants, cell surface lyric agents, therapeutic genes, plasmids comprising therapeutic genes, Cox II inhibitors, RNA-polymerase inhibitors, cyclooxygenase inhibitors, steroids, and NSAIDs (nonsteroidal anti inflammatory agents).

Specific examples of therapeutic agents include: Topoisomerase I-inhibiting camptothecins and their analogs or derivatives, such as SN-38 ((+)-(4S)-4,11-diethyl-4,9-dihydroxy-IH-pyrano[3′,4′;6,7]-14 indolizine[1,2-b]quinoline-3,14(4H,12H)-dione); 9-aminocamptothecin; topotecan (hycamtin; 9-dimethyl-aminomethyl-10-hydroxycamptothecin); irinotecan (CPT-11; 7-ethyl-10-[4-(1-piperidino)-1-piperidino]-carbonyloxy-camptothecin), which is hydrolyzed in vivo to SN-38); 7-ethylcamptothecin and its derivatives (Sawada, S. et al., Chem. Pharm. Bull., 41(2):310-313 (1993)); 7-chlorornethyl-10,11-methylene dioxy-camptothecin; and others (SN-22, Kunimoto, T. et al., J. Pharmacobiodyn., 10(3): 148-151 (1987); N-formylamino-12,13,dihydro-1,11-dihydroxy-13-(beta-D glucopyransyl)-SH-indolo[2,3-a]pyrrolo[3,4-c]carbazole-5,7(6H)-dione (NB-506, Kanzawa, G. et al., Cancer Res., 55(13):2806-2813 (1995); DX-8951f and lurtotecan (GG-211 or 7-(4-methylpiperazino-methylene)-10, 11-ethylenedioxy-20(S) camptothecin) (Rothenberg, M. L., Ann. Oncol., 8(9) :837-855 (1997)); 7-(2-(N isopropylamino)ethyl)-(20S)-camptothecin (CKD602, Chong Kun Dang Corporation, Seoul Korea); BN 80245, a beta hydroxylactone derivative of camptothecin (Big, C H. et al., Biorganic & Medicinal Chemistry Letters, 7(17): 15 2235-2238, 1997);

Other examples of therapeutic agents include topoisomerase I/II-inhibiting compounds such as 6-[[2-dimethylamino) ethyl]amino]-3-hydroxy-7H-indeno[2,1-c]quinolin-7-one dihydrochloride, (TAS 103, Utsugi, T., et al., Jpn. J. Cancer Res., 88(10):992-1002 (1997)); 3-methoxy 1 IH-pyrido[3′,4′-4,5]pyrrolo [3,2-c]quinoline-1,4-dione (AzalQD, Riou, J. F., et al., 20 Mol. Pharmacol., 40(5):699-706 (1991)); Anthracyclines such as doxorubicin, daunorubicin, epirubicin, pirarubicin, and idarubicin; Vinca alkaloids such as vinblastine, vincristine, vinleurosine, vinrodisine, vinorelbine, and vindesine; Platinum compounds such as cisplatin, carboplatin, ormaplatin, oxaliplatin, zeniplatin, enloplatin, lobaplatin, spiroplatin, ((−)—(R)-2-aminomethylpyrrolidine (1,1-cyclobutane dicarboxylato)platinum), (SP-4-3(R)-1,1-cyclobutane dicarboxylato(2-)-(2-methyl-1,4-butanediamine-N,N7) platinum), nedaplatin, and (bis-acetato-ammine-dichloro-cyclohexylamine-platinum(IV)); Anti-microbial agents such as gentamicin and nystatin; Quinazoline antifolates thymidylate synthase inhibitors such as described by Hennequin et al. Quinazoline Antifolates Thymidylate Synthase Inhibitors: Lipophilic Analogues with Modification to the C2-Methyl Substituent (1996) J. Med. Chem. 39, 695-704; Growth factor receptor inhibitors such as described by: Sun L. et al., Identification of Substituted 3-[(4,5,6,7-Tetrahydro-IH-indol-2-yl)methylene]-1,3 dibydroindol-2-ones as Growth Factor Receptor Inhibitors for VEGF-R2 (Flk 1/KDR), FGF-R1, and PDGF-Rbeta Tyrosine Kinases (2000) J. Med. Chem. 43:2655-2663; and Bridges A. J. et al. Tyrosine Kinase Inhibitors. 8. An Unusually Steep Structure-Activity Relationship for Analogues of 4-(3-Bromoanilino)-6,7 dimethoxyquinazoline (PD 153035), a Potent Inhibitor of the Epidermal Growth Factor Receptor (1996) J. Med. Chem. 39:267-276, Inhibitors of angiogenesis, such as angiostatin, endostatin, echistatin, thrombospondin, plasmids containing genes which express anti-angiogenic proteins, and methionine aminopeptidase-2 inhibitors such as fumagillin, TNP-140 and derivatives thereof; and other therapeutic compounds such as 5-fluorouracil (5-FU), mitoxanthrone, cyclophosphamide, mitomycin, streptozocin, mechlorethamine hydrochloride, melphalan, cyclophosphamide, triethylenethiophosphoramide, carmustine, lomustine, semustine, hydroxyurea, thioguanine, decarbazine, procarbazine, mitoxantrone, steroids, cytosine arabinoside, methotrexate, aminopterin, motomycin C, demecolcine, etopside, mithramycin, Russell's Viper Venom, activated Factor IX, activated Factor X, thrombin, phospholipase C, cobra venom factor [CVF], and cyclophosphamide.

In particular embodiments of the present invention, the therapeutic agent is selected from: a) an antineoplastic agent, e.g., camptothecin or an analog thereof, such as topotecan doxorubicin, daunorubicin, vincristine, mitoxantrone, carboplatin and RNA-polymerase inhibitors, especially camptothecin or analogs thereof, and more especially topotecan; b) an anti-inflammatory agent, e.g., cyclooxygenase inhibitors, steroids, and NSAIDs; c) an anti-angiogenesis agent, e.g., fumagillin, tnp-140, cyclooxygenase inhibitors, angiostatin, endostatin, and echistatin; d) anti-infectives; and e) combinations thereof.

Examples of diagnostic agents include contrast agents for imaging including paramagnetic, radioactive or fluorogenic ions. Specific examples of such diagnostic agents include those disclosed in U.S. Pat. No. 5,855,866 issued to Thorpe et al. on Jan. 5, 1999.

Such agents can be associated with the inner core of the polymeric micelles by specific interactions such as hydrogen bonding, electrostatic and or ionic interactions. These interactions are facilitated by the introduction of sulfonic acid groups into the hydrophobic segments of the amphiphilic block copolymer.

Polymeric micelles can be prepared from the amphiphilic copolymer as the polymer component. Methods of making polymeric micelles are well known in the art, e.g., as described in M. C. Jones and J. C. Leroux, European Journal of Pharmaceutics and Biopharmaceutics, 48 (1999), 101-111.

In general, polymeric micelles are formed by dissolving a lyophilized powder of the amphiphilic polymer at a concentration greater than its critical micelle concentration (cmc), the micelles being formed by a spontaneous self-assembly process. Such micelles will have a hydrophobic core and hydrophilic outer domain. The inventive polymers of this invention, having benzoyl sulfonic acid groups, also spontaneously form polymeric micelles by dissolving a lyophilized powder of the complex at a concentration greater than the cmc of the complex. The micelles have a hydrophobic core and a hydrophilic outer domain. In preferred embodiments, where the cationic bioactive agent is complexed the hydrophobic terminus of the amphiphilic polymeric copolymer, such that after micellation the bioactive agent is present in the inner core of the polymeric micelle.

Indications to which the present invention may be applied include but are not exclusive of processes characterized by angiogenesis, e.g., inflammation processes as in osteo and rhumatoid arthritis, diabetic retinopathy, hemangiomas, psoriasis and cancerous tumors (solid primary tumors as well as metastatic disease).

Polymeric micelles are administered to a patient, typically intravenously. The vehicles are carried by the circulatory system to the targeted tissue, where the vesicle associates with the tissue, tumor to inhibit tumor growth or metastasis. In addition, the agent associated with the vesicle may be released or may diffuse to the targeted tissue. For example, a chemotherapeutic agent may treat a tumor or a contrast agent may serve to provide contrast for imaging purposes.

### EXAMPLES

Materials and Methods

Polycaprolactone (Mn=1250), Methoxypolyethyleneglycol (Mn=2000), Sulfobenzoic anhydride and Stannous Octoate were all obtained from Aldrich Chemical Company (MO, USA). DL-lactide was purchased from Purac (IL, USA).

The molecular weights of the polymers were determined by a Shimadzu GPC system consisting of a Shimadzu LC-10AD Pump, SIL-10AXL Autosampler, SPD-10A UV detector, a Waters 2410 refractive Index detector, and a Viscotek T60A dual detector. Data acquisition and processing is performed by a Viscotek Trisec GPC 3.0 software using universal calibration mode.

The percentage functionalization is determined by acidimetric titration, and by taking into account the Mn (number average molecular weight determined by GPC) and theoretical number of end groups per chain. About 0.2 g of the polymer was accurately weighed and dissolved in milliQ water. This solution was titrated against 0.01N sodium hydroxide solution using phenolphthalein as the indicator.

Critical Micelle Concentration (cmc) was determined by a Kruss K12 Tensiometer using the Wilhemy plate method. Data acquistion and processing was done using K122 software. A polymer solution of known concentration was automatically titrated into the milliQ water. The surface tension values were automatically recorded and plotted against respective concentration to yield the cmc. Size of the polymeric micelles were determined by a Malvern 5000 Zeta Sizer at a polymer concentration in water above the cmc.

1) Functionalization of Polycaprolactonediol with Benzoyl Sulfonic Acid Groups

Thirty grams (30 g) of polycaprolactone diol (Aldrich) was dried by azeotropic distillation under toluene using a Dean-Stark Apparatus. The residual toluene was removed under vacuum.

Ten grams (10 g) of the dried polycaprolactone diol, 1.43 g of sulfobenzoic anhydride (Aldrich) and 0.1 mL of 0.2M solution of stannous octoate (Aldrich) in toluene, were added to a flame dried three necked 250 mL round bottom flask. The contents were heated at 160 ° C. and stirred for 1 hour, under dry nitrogen atmosphere. The flask was cooled and the contents was dissolved in 10 mL acetone. This acetone solution was added to 100 mL of cold 1:1 mixture of isopropanol and hexane, resulting in a cloudy solution. This cloudy solution was centrifuged and the residue was collected. The residue was suspended in milliQ water and lyophilized. Yield 6 g.

The extent of functionalization was 96%, as determined by acidimetric titration.

2) Synthesis of Poly(Lactide-Block-Methoxypolyethylene Glycol)

Fifty grams (50 g) of methoxypolyethylene glycol (Aldrich, Mn=2000), was dried by azeotropic distillation under toluene using a Dean-Stark Apparatus. The residual toluene was removed under vacuum.

In a dry box filled with dry nitrogen, 40 g of the dried methoxypolyethylene glycol and 50 g of dl-lactide (Purac) were weighed out into glass reactor. The reactor was sealed and transferred to an oil bath in a chemical hood. The reactor was evacuated three times and purged with dry nitrogen. 0.5 ml of 0.01M stannous octoate in dry toluene was added to the reactor using a syringe. The reactor was put under vacuum and then purged with dry nitrogen gas three times. The reactor was immersed in an oil bath at 160° C. The contents were mixed with a mechanical stirrer. Polymerization was continued for 6h at 160° C. The copolymer was collected after cooling the reaction mixture.

Nine grams of the polymer from example 2 was dissolved in 50 ml of acetone. The acetone solutions were separately added to 700 ml isopropanol. Cloudy solutions obtained were centrifuged. Residues were collected, dissolved in 20ml of water and lyophilized. Mn determined by GPC was 3500.

3) Functionalization of the Block Copolymer from Example 2 with Benzoyl Sulfonate Groups.

Ten grams (10 g) of the crude block copolymer from example 2 was placed in a 250 ml three necked round bottom flask purged with dry nitrogen, to which was added 0.5 g of sulfobenzoic anhydride and 0.1 mL of 0.2M stannous octoate in toluene solution were added to the flask. The flask was immersed in an oil bath kept at 160° C. The reaction mixture was stirred, with heating, for one hour. The flask was cooled and the contents were dissolved in 50 mL acetone. The acetone solution was added to 500 mL isopropanol. The cloudy solution was centrifuged to collect the residue. The residue was suspended in milliQ water and lyophilized. Yield 6.5 g.

The percentage functionalization was 70%, as determined by acidimetric titration. The critical micelle concentration was 0.015 mg/mL. The mean particle size was 155 nm.

4) Synthesis of Poly(Lactide-Block-Methoxypolyethylene Glycol) and Functionalization in One Step.

In a dry box filled with dry nitrogen, 4 g of dried methoxypolyethylene glycol dried in example 2 and 6 g of dl-Lactide were weighed into a flame dried three necked 250 mL round bottom flask. The round bottom flask was sealed and transferred to a chemical hood. The flask was immersed in an oil bath, and evacuated and purged three times with dry nitrogen. Stannous octoate (0.5 ml of a 0.01 M solution in dry toluene) was added to the flask using a syringe. The flask was put under vacuum and then purged with dry nitrogen gas three times. The temperature of the oil bath was raised to 160° C. The contents was stirred and the polymerization was continued for 6h at 160° C. under dry nitrogen atmosphere. Upon completion of the polymerization, 0.1 g of sulfobenzoic anhydride was added and the reaction was continued for 1 h at 160° C. Then the flask was cooled and the contents dissolved in 25 mL acetone. The acetone solution was added to 300 mL isopropanol to give a cloudy solution, which was centrifuged to collect the residue. The residue was suspended in 20 mL water and lyophilized.

5) Preparation of Complex of Topotecan and Sulfonated Derivatized PEG-PLA

A portion (100.1 mg) of sulfonate functionalized PEG-PLA (from Example 3) was dissolved in 2 mL methanol to form a clear solution. A solution of topotecan HCl (7.26 mg) in 3.0 mL of 1:1 methanol and methylene chloride was added to the polymer in methanol solution. The mixture was stirred for 3 hr, concentrated under vacuum to 1.5 mL, then precipited in cold isopropanol (40 mL). The powder was collected by centrifugation and washed first with 5 mL of a mixed solvent containing 60% isopropanol and 40% hexane, followed by 5 mL hexane and dried under nitrogen. The drug content was analyzed by HPLC equiped with a size exclusion column and a diode array detector. The ratio of drug and polymer in weight was 1.4-2.1%, and the loading efficiency was 14-22%.

6) Preparation of Complex of Topotecan and Sulfonated Derivatized PEG-PLA

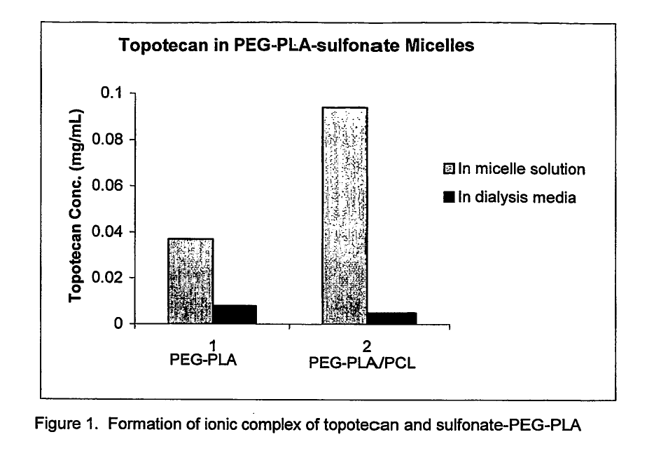

Sulfonate functionalized PEG-PLA (72 mg) (from Example 3) was dissoved in methanol (1 mL) forming a solution with a concentration of 72 mg/mL. Topotecan HCl (5 mg) in methanol (1 mL) solution was added to the polymer solution. Stirred for 24 h, the mixture was dialyzed against water (300 mL), and the media was replaced once with deionized water. After 96 h, the final concentration in the dialysis bag and in media was analyzed by UV-Vis, free topotecan was used as standard. In parallel, a formulation containing PEG-PLA-sulfonate (72 mg) (from Example 3) and polycaprolactone sulfonate (16 mg, from Example 1) was prepared. The concentration difference of topotecan between in micelle solution and in dialysis media was compared in FIG. 1.

7) Preparation of Polymeric Micelles from Topotecan and Polymer Complex

Sulfonate derivatized PEG-PLA (108 mg) (from Example 3) was converted to its sodium salt by titrating its aqueous solution with saturated sodium bicarbonate. A white powder was obtained after the polymer solution was lyophilized for 24 hr. To prepared the topotecan complex, a solution of topotecan HCl salt (7.0 mg) in methanol (2mL) was added to the polymer in methanol (3 mL) and CH₃CN (2 mL) solution. The mixture was stirred for 40 min and was sampled (0.05 mL) and assayed by SEC-HPLC. The remaining mixture was rotavaped to completely remove solvents. Methylene chloride (3 mL) was added to the mixture to extract soluble topotecan-polymer complex from free topotecan HCl. The liquid phase was separated and added in dropwise to a phosphate buffer solution for analysis by SEC-HPLC. The drug content in the complex was 1.4% and loading efficiency was 13.4%.

8) Formulation Topotecan with PEG-PLA Sulfonate and Evaluation of Release

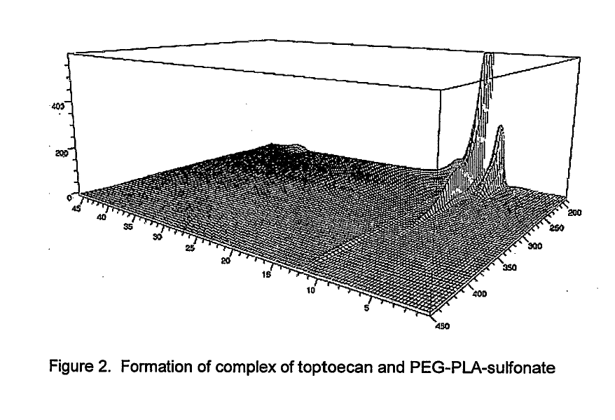

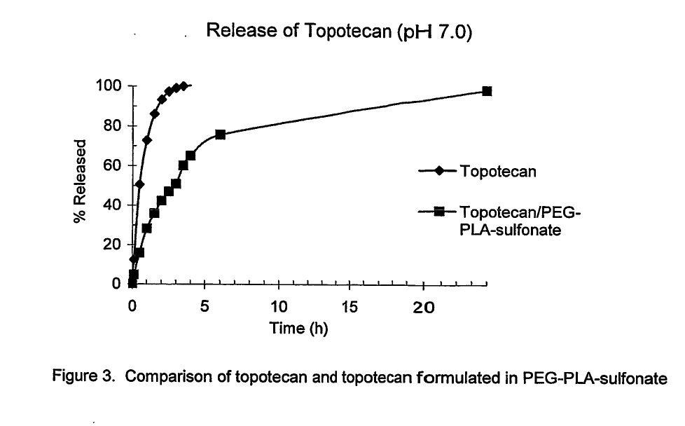

To a polymer solution in methanol (50 mg/mL) added topotecan HCl in 1:1 methanol:methylene chloride solution. The initial drug/polymer ratio was in the range of 2-15%. The mixture was stirred for 40 min, followed by adding it slowly to a phosphate buffer solution (pH 6.0). The organic solvents were slowly evaporated under vacuum under magnetic stirring, and the mixture was then transferred to a dialysis tube made of regenerated cellulose and having 3500 molecular weight cut off (VWR International, Bridgeport, N.J.) The dialysis tube was placed in a PBS buffer (pH 7.4). Samples were taken from the medium at predetermined time intervals and were injected to a SEC-HPLC system to analyze the drug content. Micelle formation was observed from the chromatographs (FIG. 1) and drug release was shown in FIG. 2 and FIG. 3.

9) Formulation Topotecan with PEG-PLA Sulfonate in the Presence of Excipients

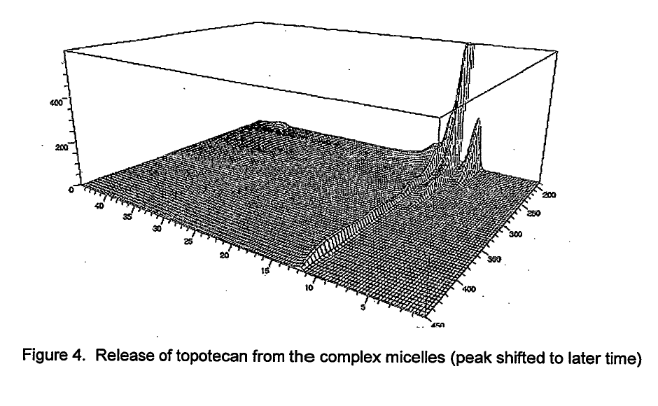

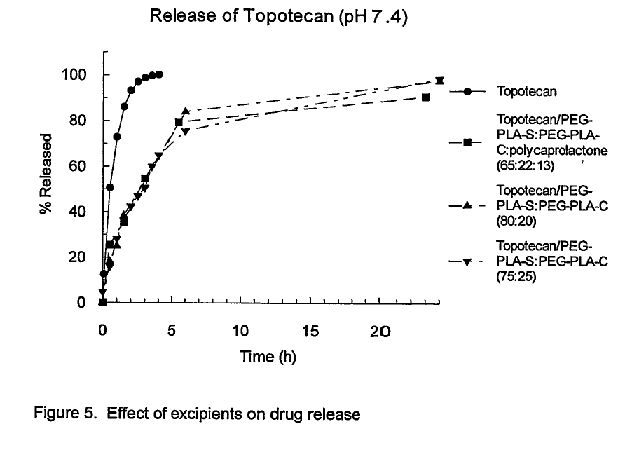

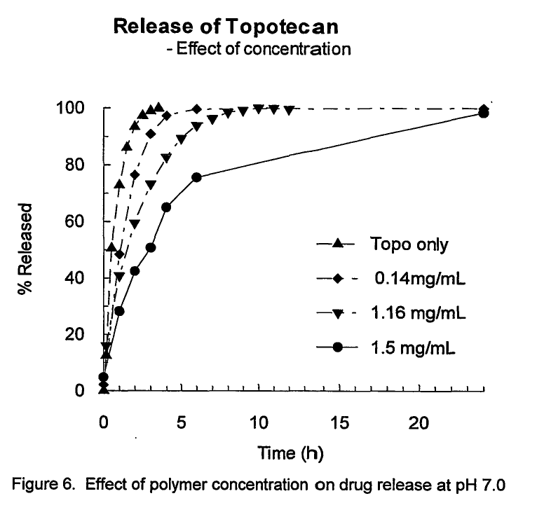

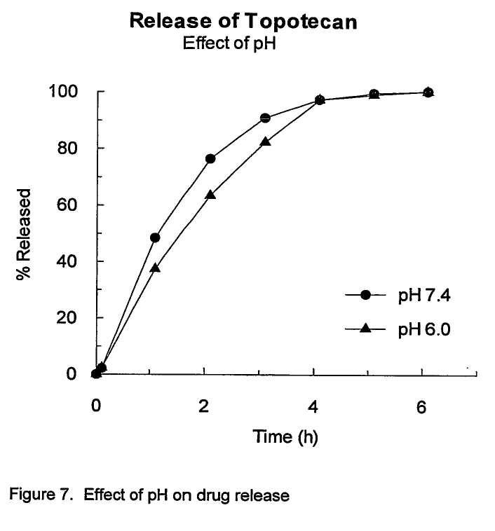

PEG-PLA sulfonate (100 mg) and an excipient (20 mg) were dissolved in methanol (2 mL). The weight ratio of polymer and excipient was adjusted in the range of 5-25%. To the polymer solution was added a solution of topotecan HCl (5 mg) in 1:1 methanol:methylene chloride (2 mL). The initial ratio of polymer and drug was from 2% to 10%. The mixture was stirred for40 min, followed by adding it slowly to a phosphate buffer (pH 6.0). The organic solvents were slowly evaporated under vacuum under magnetic stirring, and the mixture was then transferred to a dialysis tube made of regenerated cellulose and having 3500 molecular weight cut off The dialysis tube was placed a PBS buffer (pH 7.4). Samples were taken from the medium at predetermined time intervals and injected to a SEC-HPLC system to analyze the drug content. Drug release was shown in FIG. 4. Effect of polymer concentrations and pH on drug release was shown in FIG. 5 and 6.

10) In Vivo Evaluation of Polymeric Micelles from Topotecan Complex of Sulfonate Derivatized PEG-PLA in Rats.

A formulation of topotecan and sulfonate derivatized PEG-PLA was prepared by solvent evaporation procedure. The formulation contained 200 mg of PEG-PLA-sulfonate, 12.5 mg PEG-PLA-COOH, and 11.25 mg topotecan HCl. The solution was lyophilized and kept as a powder. For dosing, the powder was dissolved in saline solution and administered to rats at a dose of 5 mg/kg. Blood samples were taken at a predetermined time intervals and the serum samples were assayed by HPLC. Topotecan HCl in saline solution (1.5 mg/mL) was dosed as a control.

The subject invention is not limited to the particular embodiments described hereinabove, but includes all modifications thereof within the scope of the appended claims and their equivalents. Those skilled in the art will recognize through routine experimentation that various changes and modifications can be made without departing from the scope of this invention. All documents cited or referred to herein, including issued patents, published and unpublished patent applications, and other publications are hereby incorporated herein by reference as though fully set forth.

1. A complex of an amphiphilic copolymer with a bioactive agent, wherein the amphiphilic copolymer has benzoyl sulfonic acid groups on the hydrophobic segment of said copolymer.

2. A complex according claim 1, wherein said complex forms micelles in aqueous media.

3. A complex according to claim 1, wherein the amphiphilic copolymer is comprised of a hydrophilic polymer selected from the group consisting of: a polyalkylether, dextran, dextran, carboxymethyldextran, dextran sulfate, aminodextran, cellulose, carboxymethyl cellulose, chitin, chitosan, succinyl chitosan, carboxymethylchitin, carboxymethylchitosan, hyaluronic acid, a starch, an alginate, chondroitin sulfate, albumin, pullulan, carboxymethyl pullulan, polyglutamic acid, polylysine, polyaspartic acid, HPMA, styrene maleic anhydride copolymer, divinylethyl ether maleic anhydride copolymer, polyvinyl pyrrolidone, and polyvinylalcohol.

4. A complex according to claim 1, wherein the amphiphilic polymer is a block copolymer made of hydrophilic and hydrophobic polymers.

5. A complex according to claim 4, wherein the hydrophilic polymer is polyoxyethylene glycol, polyoxypropylene glycol, polyoxyethylene/propylene glycol, dextran, carboxymethyldextran, dextran sulfates, aminodextran, cellulose, carboxymethyl cellulose, chitin, chitosan, succinyl chitosan, carboxymethylchitin, carboxymethylchitosan, hyaluronic acid, a starch, an alginate, chondroitin sulfate, albumin, pullulan, carboxymethyl pullulan, polyglutamic acid, polylysine, polyaspartic acid, HPMA, styrene maleic anhydride copolymer, divinylethyl ether maleic anhydride copolymer, polyvinyl pyrrolidone, and polyvinylalcohol.

6. A complex according to claim 5, wherein the hydrophilic polymer is polyethylene glycol.

7. A complex according to claim 6, wherein the polyethylene glycol has a molecular weight of about 1000-10000.

8. A complex according to claim 1, comprising a hydrophobic polymer, wherein the hydrophobic polymer is selected form a poly(alpha-hydroxy acid), polydioxanone, a polycarbonate, a polyanhydride, a polyorthoester, and a hydrophobic derivative of a poly(alpha-amino acid).

9. A complex according to claim 8, wherein the hydrophobic polymer is polylactic acid.

10. A complex according to claim 1, wherein the bioactive agent is selected from the group consisting of topotecan, doxorubicin, adriamycin, vincristine, cisplatin, and a combination thereof.

11. A complex according to claim 1, wherein the bioactive agent is topotecan.

12. A method of treating a cancer comprising administering an effective amount of the complex according to claim 1 to a patient in need thereof.

13. A method of treating osteo arthritis, rheumatoid arthritis, diabetic retinopathy, hemangiomas or psoriasis comprising administering an effective amount of the complex according to claim 1 to a patient in need thereof.

14. A complex of an amphiphilic copolymer with a contrast agent, wherein the amphiphilic copolymer has benzoyl sulfonic acid groups on the hydrophobic segment of said copolymer.

15. A method of diagnostic imaging comprising administering an effective amount of the complex according to claim 14 to a patient in need thereof.

16. A process of making an amphiphilic copolymer having benzoyl sulfonic acid groups by reacting the amphiphilic copolymer with sulfobenzoic anhydride either in the melt or in solution.
